(19) United States
(12) Patent Application Publication
(10) Pub. No.: US 2025/0109394 A1
(43) Pub. Date: Apr. 3, 2025

(54) MOVING MAGNET FOR MAGNETIC BEAD-ASSISTED SEPARATION

(71) Applicant: Volta Labs, Inc., Boston, MA (US)

(72) Inventor: Udayan UMAPATHI, Somerville, MA (US)

(21) Appl. No.: 18/980,985

(22) Filed: Dec. 13, 2024

Related U.S. Application Data
(63) Continuation of application No. PCT/US2023/068630, filed on Jun. 16, 2023.
(60) Provisional application No. 63/353,474, filed on Jun. 17, 2022.

(51) Int. Cl. C12N 15/10; B01L 3/00; B03C 1/01; B03C 1/28

(52) U.S. Cl. CPC C12N 15/1013; B01L 3/502792; B03C 1/01; B03C 1/288; B01L 2200/0668; B01L 2400/0427; B01L 2400/043; B03C 2201/18

(57) ABSTRACT

Described herein are methods, systems, and devices for providing said droplet on a surface, wherein said surface is adjacent to a magnet configured to provide a magnetic field contacting said droplet, and wherein said droplet comprises one or more artifacts that are responsive to said magnetic field, displacing said magnet proximal to said droplet, wherein said magnetic field contacting said droplet comprises a flux density of at least about 4 millitesla (“mT”) to at least about 10 mT, thereby manipulating said one or more artifacts that are responsive to said magnetic field.

### CROSS-REFERENCE

This application is a continuation of International Application PCT/US2023/068630, filed Jun. 16, 2023, which claims benefit of U.S. Provisional Application No. 63/353,474, filed Jun. 17, 2022, which are herein incorporated by reference in their entirety.

### BACKGROUND

Many biological protocols rely on the use of magnetic beads to perform operations on DNA, proteins, and other biological assets. These magnetic beads typically need to be well suspended and mixed with various biological samples. The biological asset of interest may then precipitate out of solution and become captured on the magnetic beads. At this point the beads must be separated from the rest of the solution in order to extract and purify the biological asset of interest. To do this, a magnetic field may be introduced in order to pellet the magnetic beads in a smaller area.

Current methods have not properly accounted for shear stress on capture nucleic acid molecules.

### SUMMARY

An aspect of the instant disclosure is method of processing a droplet comprising providing an electrowetting array, wherein said electrowetting array is adjacent to a magnet configured to apply a magnetic field to said electrowetting array in one or more of three directional axes, providing on said array a droplet comprising one or more artifacts that are responsive to said magnetic field, actuating said magnet in said one or more of three directional axes with respect to said array to separate said one or more artifacts from said droplet. In some embodiments, said magnet is moved parallel to said array in the x direction. In some embodiments, said magnet is moved parallel to said array in the y direction. In some embodiments, said magnet is moved orthogonally to said array. In some embodiments, the strength of said magnetic field is modulated. In some embodiments, the strength of said magnetic field is increased. In some embodiments, the strength of said magnetic field is decreased. In some embodiments, said magnet is a permanent magnet. In some embodiments, said magnet is an electromagnet or an electro-permanent magnet. In some embodiments, said electromagnet or said electro-permanent magnet modulates the strength of said magnetic field in a time-dependent manner. In some embodiments, said magnet is positioned above said electrowetting array. In some embodiments, said magnet moves orthogonally with respect to said electrowetting array to separate said one or more artifacts from said droplet. In some embodiments, said magnet is positioned below said electrowetting array.

Another aspect of the instant disclosure is a method of removing one or more artifacts from a droplet comprising providing a magnet configured to apply a magnetic field to said droplet and actuating said magnet with respect to said droplet to separate said one or more artifacts from said droplet, wherein said droplet is less than 40 microliters. In some embodiments, said magnet is moved parallel to said array in the x direction. In some embodiments, said magnet is moved parallel to said array in the y direction. In some embodiments, said magnet is moved orthogonally to said array. In some embodiments, the strength of said magnetic field is modulated. In some embodiments, the strength of said magnetic field is increased. In some embodiments, the strength of said magnetic field is decreased. In some embodiments, said magnet is a permanent magnet. In some embodiments, said magnet is an electromagnet or an electro-permanent magnet. In some embodiments, said electromagnet or said electro-permanent magnet modulates the strength of said magnetic field in a time-dependent manner. In some embodiments, said magnet is positioned above said electrowetting array. In some embodiments, said magnet moves orthogonally with respect to said electrowetting array to separate said one or more artifacts from said droplet. In some embodiments, said magnet is positioned below said electrowetting array. In some embodiments, said droplet is less than 30 microliters. In some embodiments, said droplet is less than 20 microliters. In some embodiments, said droplet is less than 10 microliters.

Another aspect of the instant disclosure is a method for processing a droplet, the method comprising: providing the droplet on a surface, wherein the surface is adjacent to a magnet configured to provide a magnetic field contacting the droplet, and wherein the droplet comprises one or more artifacts that are responsive to the magnetic field; displacing the magnet proximal to the droplet, wherein the magnetic field contacting the droplet comprises a flux density of at least about 4 millitesla (“mT”) to at least about 10 mT, thereby manipulating the one or more artifacts that are responsive to the magnetic field. In some embodiments, the magnet is displaced along an axis orthogonal to the surface. In some embodiments, the magnet is displaced at a distance of about 0 millimeters to about 15 millimeters from the surface. In some embodiments, the surface comprises an electrowetting array. In some embodiments, the one or more artifacts that are responsive to the magnetic field comprise a nucleic acid molecule. In some embodiments, the one or more artifacts that are responsive to the magnetic field comprise a plurality of nucleic acid molecules. In some embodiments, the magnet is displaced along an axis parallel to the surface. In some embodiments, the method further comprises motioning the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters. In some embodiments, the nucleic acid molecule comprises at least 100 kb. In some embodiments, at least 20% of the plurality of nucleic acid molecules comprise at least 100 kb. In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the flux density is sufficient for immobilizing the one or more artifacts that are responsive to the magnetic field during one or more droplet operations. In some embodiments, the one or more droplet operations comprise agitation of the surface.

Another aspect of the instant disclosure is a method for processing a droplet, the method comprising: providing the droplet on a surface, wherein the surface is adjacent to a magnet configured to provide a magnetic field contacting the droplet, and wherein the droplet comprises one or more artifacts that are responsive to the magnetic field; displacing the magnet proximal to the droplet at a distance of about 0 millimeters to about 15 millimeters from the surface, thereby manipulating the one or more artifacts that are responsive to the magnetic field. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a nucleic acid molecule. In some embodiments, the one or more artifacts that are responsive to the magnetic field comprise a plurality of nucleic acid molecules. In some embodiments, the magnet is displaced along an axis parallel to the surface. In some embodiments, the method further comprises motion the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters. In some embodiments, the nucleic acid molecule comprises at least 100 kb. In some embodiments, at least 20% of the plurality of nucleic acid molecules comprise at least 100 kb. In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the distance is sufficient for immobilizing the one or more artifacts that are responsive to the magnetic field during one or more droplet operations. In some embodiments, the one or more droplet operations comprise agitation of the surface.

Another aspect of the instant disclosure is a method for processing a droplet, the method comprising: providing the droplet on a surface, wherein the surface is adjacent to a magnet configured to provide a magnetic field contacting the droplet, wherein the droplet comprises one or more artifacts that are responsive to the magnetic field, and wherein the one or more artifacts that are responsive to the magnetic field comprise a nucleic acid molecule; motioning the magnet and maintaining the nucleic acid molecule at 100 kb or more, thereby manipulating the one or more artifacts that are responsive to the magnetic field. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the method further comprises motion the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters. In some embodiments, at least 20% of the plurality of nucleic acid molecules comprise at least 100 kb. In some embodiments, the method further comprises maintaining at least 20% the plurality of nucleic acid molecules at 100 kb or more. In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the distance is sufficient for immobilizing the one or more artifacts that are responsive to the magnetic field during one or more droplet operations. In some embodiments, the one or more droplet operations comprise agitation of the surface.

Another aspect of the instant disclosure is a system for processing a droplet, the system comprising: a surface configured to support a droplet; a magnet adjacent the surface, wherein the magnet is configured to provide a magnetic field contacting the droplet, wherein the droplet comprises one or more artifacts that are responsive to the magnetic field, and wherein the one or more artifacts that are responsive to the magnetic field comprise a nucleic acid molecule; a controller mechanically coupled to the magnet, wherein the controller is configured to displace the magnet proximate to the droplet and maintaining the nucleic acid molecule at 100 kb or more. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the controller is configured maintain a flux density of the magnetic field of at least about 4 millitesla (“mT”) to at least about 10 mT of the magnetic field contacting the droplet. In some embodiments, the controller is configured to maintain the magnet a distance of about 0 millimeters to about 15 millimeters from the surface. In some embodiments, the droplet is less than 30 microliters. In some embodiments, the droplet is less than 20 microliters. In some embodiments, the droplet is less than 10 microliters. In some embodiments, at least 20% of the plurality of nucleic acid molecules comprise at least 100 kb. In some embodiments, the system maintains at least 20% the plurality of nucleic acid molecules at 100 kb or more. In some embodiments, the system maintains the nucleic acid molecule at 100 kb or more. In some embodiments, the system maintains at least 20% the plurality of nucleic acid molecules at 100 kb or more. In some embodiments, the distance is sufficient for immobilizing the one or more artifacts that are responsive to the magnetic field during one or more droplet operations. In some embodiments, the one or more droplet operations comprise agitation of the surface.

Another aspect of the instant disclosure is a device for processing a droplet, the device comprising: a surface configured to support a droplet; a magnet adjacent the surface, wherein the magnet is configured to provide a magnetic field contacting the droplet, wherein the droplet comprises one or more artifacts that are responsive to the magnetic field, and wherein the one or more artifacts that are responsive to the magnetic field comprise a nucleic acid molecule; a controller mechanically coupled to the magnet, wherein the controller is configured to displace the magnet proximate to the droplet and maintaining the nucleic acid molecule at 100 kb or more. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the controller is configured maintain a flux density of the magnetic field of at least about 4 millitesla (“mT”) to at least about 10 mT of the magnetic field contacting the droplet. In some embodiments, the controller is configured to maintain the magnet a distance of about 0 millimeters to about 15 millimeters from the surface. In some embodiments, the droplet is less than 30 microliters. In some embodiments, the droplet is less than 20 microliters. In some embodiments, the droplet is less than 10 microliters. In some embodiments, at least 20% of the plurality of nucleic acid molecules comprise at least 100 kb. In some embodiments, the distance is sufficient for immobilizing the one or more artifacts that are responsive to the magnetic field during one or more droplet operations. In some embodiments, the one or more droplet operations comprise agitation of the surface.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

This specification incorporates herein by reference, in its entirety, International Application No. PCT/US2022/018549, filed on Mar. 2, 2022.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows moving the magnetic field in one or more axes aligned with the plane of the EWOD surface enables bead separations that would not be possible with EWOD alone.

FIGS. 2A-2B show modulating the strength of the magnetic field may enable reduction in shear forces when pulling magnetic beads through a supernatant droplet.

FIGS. 3A-3C show that introduction of a magnet above the droplet may be useful for separating beads from small droplets or droplets with high surface tension.

FIGS. 4A-4C depict moving the magnet to create clearance for other operations. FIG. 4A shows the magnet up close to the active surface. FIG. 4B shows the magnet has moved orthogonally to provide clearance between the magnet and surface. FIG. 4C. shows the active surface moves up and down to create agitation in the droplet surrounding the magnetic bead pellet.

FIGS. 5A-5B depict a configuration for the synthesis and assembly of biopolymers (e.g., DNA) using systems and methods described herein.

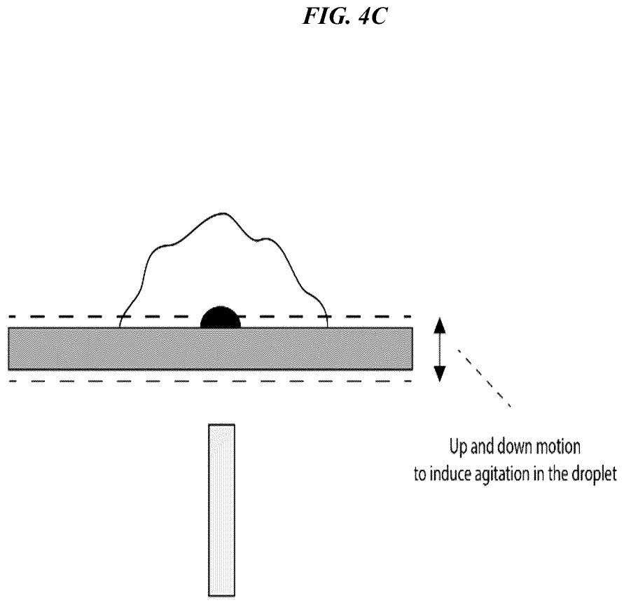

FIG. 6 depicts an example schematic Next-Generation Sequencing (NGS) workflow using systems and methods described herein. The example workflow comprises manipulating (e.g., lysing cells, digesting protein, and DNA clean-up) biological samples on an array described herein.

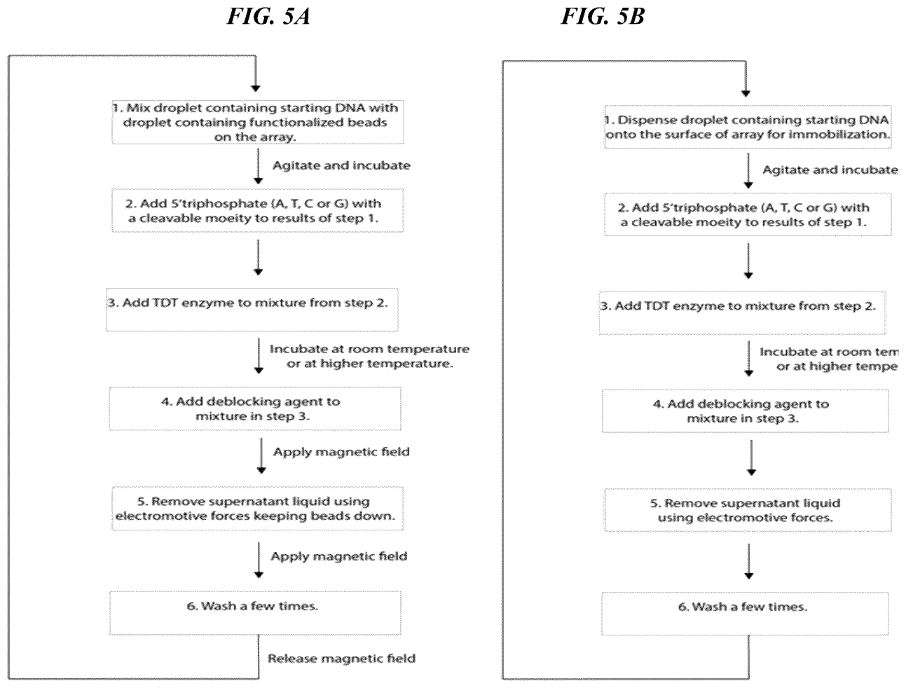

FIGS. 7A-7B illustrate one application of vibration assisted electrowetting on dielectric for the extraction of DNA using magnetic beads.

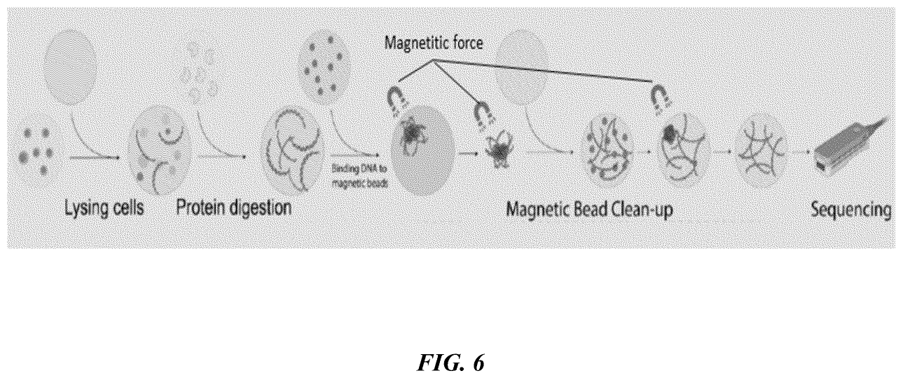

FIGS. 8A-8B show that high contact angle droplets (FIG. 8A) tend to experience greater response to vibration than droplets with lower contact angle (FIG. 8B).

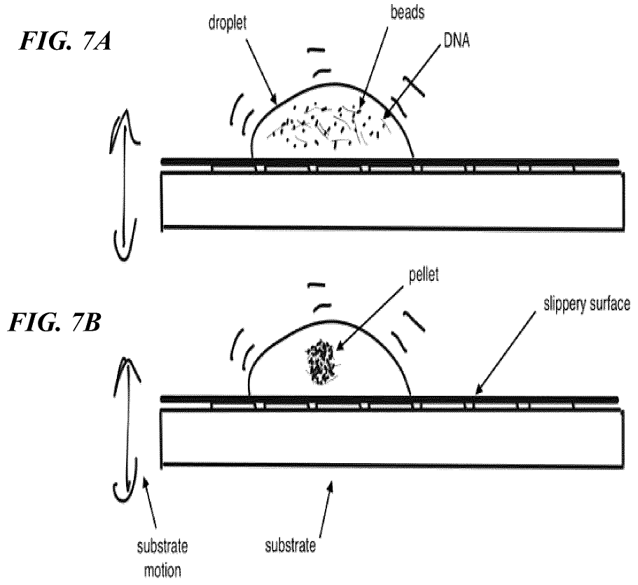

FIGS. 9A-9B depict embodiments of electro-mechanical actuators.

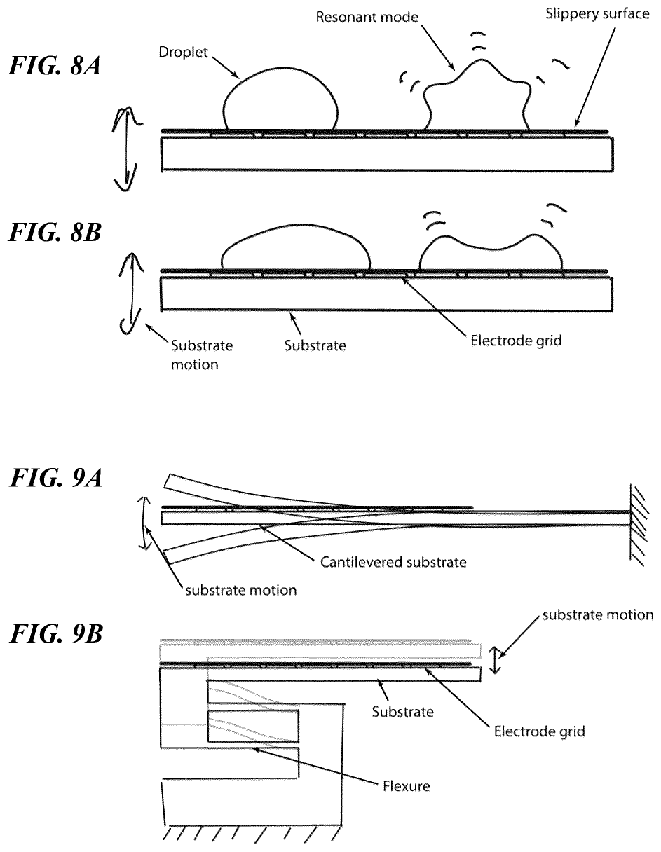

FIGS. 10A-10B depict additional embodiments of electro-mechanical actuators.

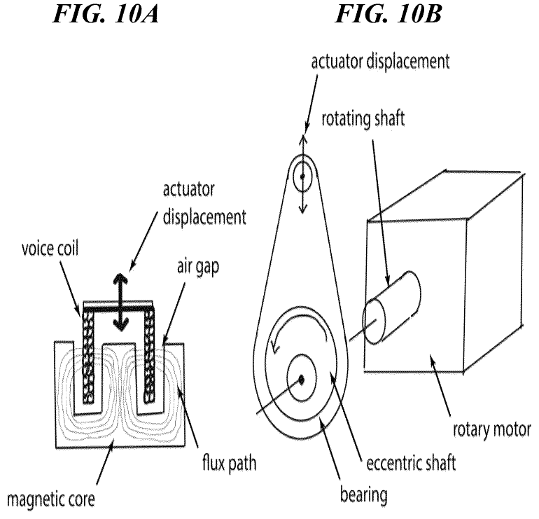

FIG. 11 shows an embodiment of efficiently coupling the actuation force of the electro-mechanical actuator into droplet vibration and, ultimately, to effective mixing.

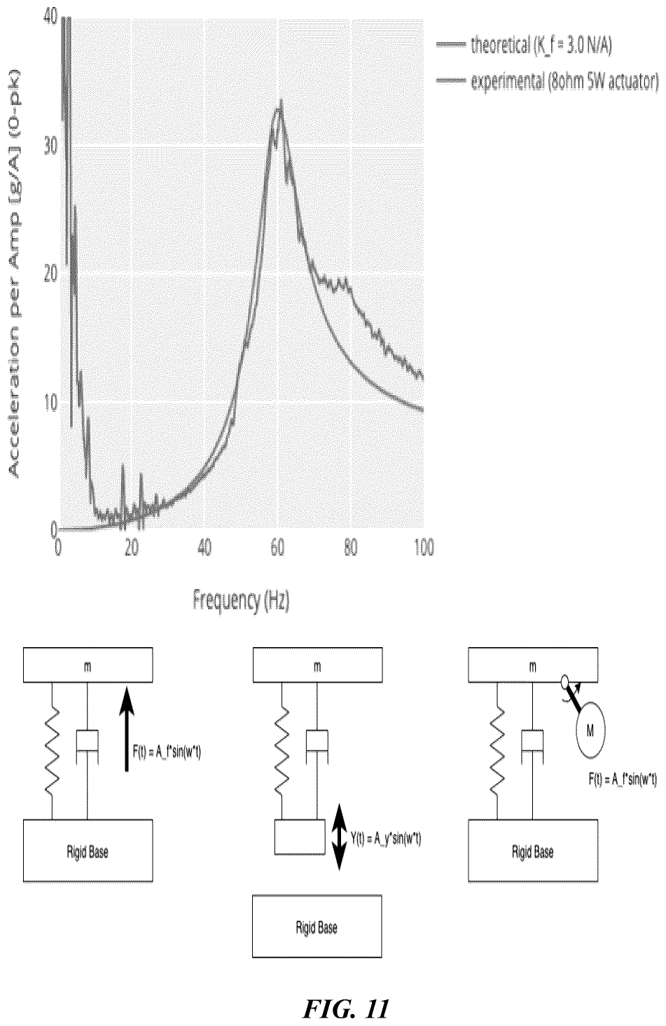

FIG. 12 shows magnet position, flux density (mT) measured at various distances from the EWOD surface.

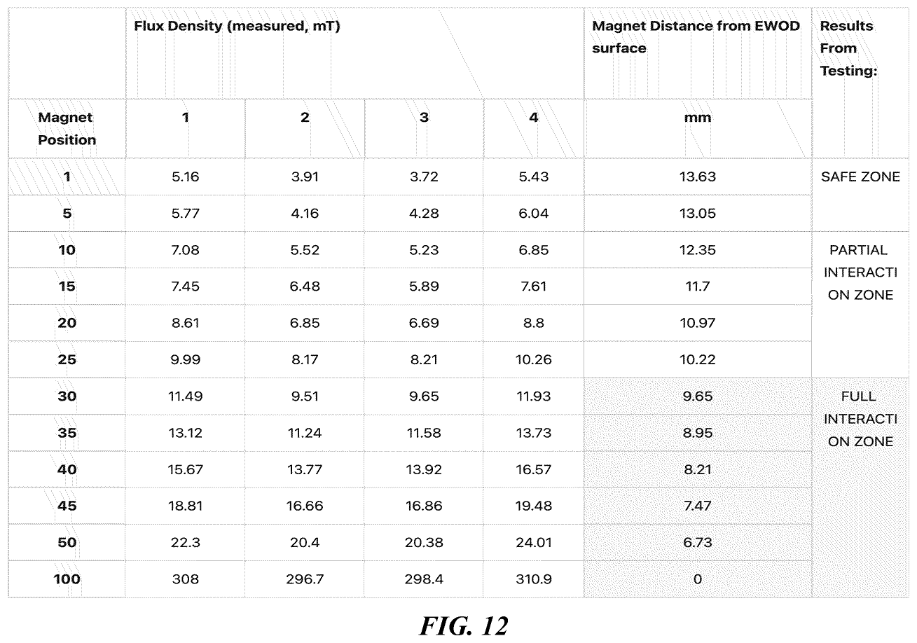

FIG. 13 shows the flux density (mT) measured on the EWOD surface as the magnet goes from position 1 to position 50. Position 1 is when the magnet is at a distance of 13.6 mm from the EWOD surface and position 50 is when the magnet is at a distance of 6.7 mm from the EWOD surface.

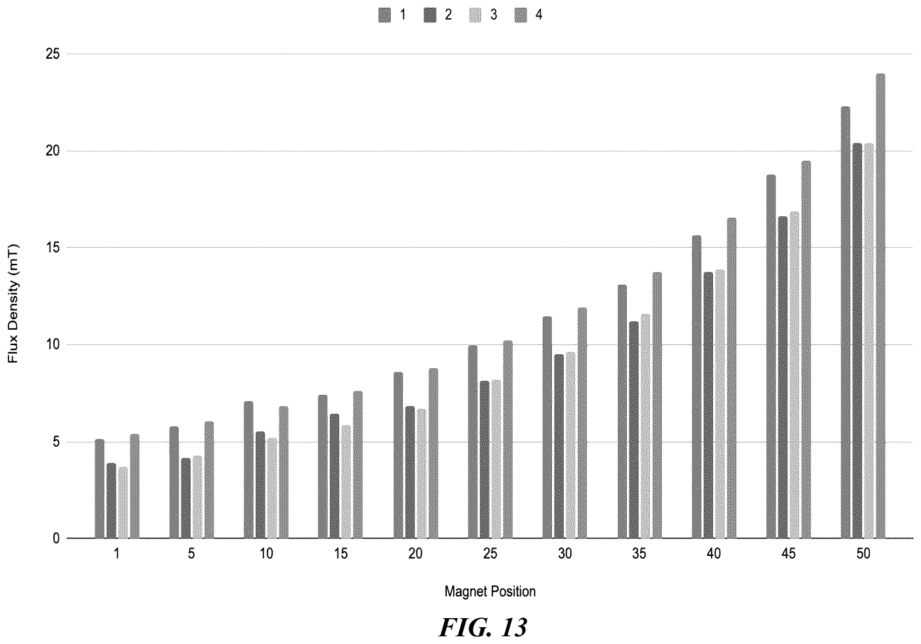

FIG. 14 represents a cartoon of computer systems used for arrays described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Many biological protocols rely on the use of magnetic beads to perform operations on DNA, proteins, and other biological assets. These magnetic beads typically need to be well suspended and mixed with various biological samples. The biological asset of interest may then precipitate out of solution and become captured on the magnetic beads. At this point the beads must be separated from the rest of the solution in order to extract and purify the biological asset of interest. To do this, a magnetic field may be introduced in order to pellet the magnetic beads in a smaller area. In prior art, the separation of the supernatant from this bead pellet is performed through the use of electrowetting based droplet operations to translate the droplet solution off of the bead pellet.

However, this approach can cause challenges in certain scenarios. For example, if the droplet volume is relatively large and/or has a low surface tension, it may form a puddle rather than a spherical cap-shaped droplet. Furthermore, because the electrowetting force can only be applied at the perimeter of the droplet/puddle, a puddle can be difficult to adequately translate using EWOD to separate it from a bead pellet (since the area in contact with the EWOD surface is large compared to the perimeter).

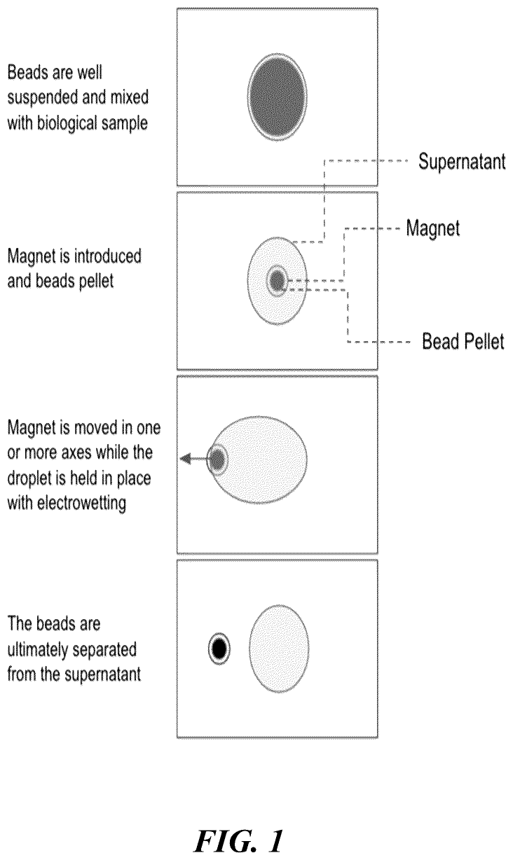

An alternative approach, described here and pictured in FIG. 1, involves positioning the magnetic field rather than using EWOD to position the supernatant. In this approach the friction/shear force between the droplet and the surface is useful to resist the force of the bead pellet as it is pulled out of the droplet. By moving the magnetic field in at least one axis aligned with the plane of the EWOD surface, it's therefore possible to perform separations between bead pellets and supernatants that wouldn't be possible with EWOD alone.

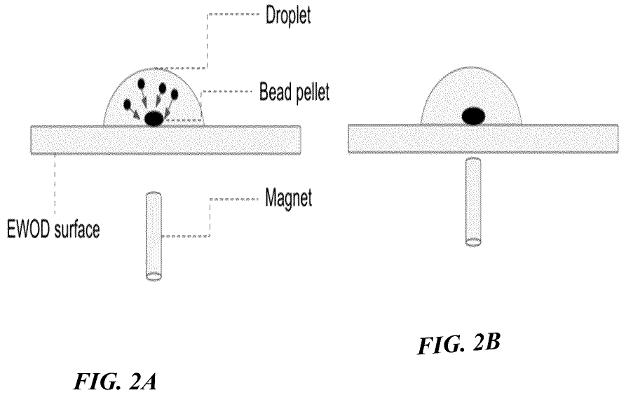

The addition of control over the magnetic field strength may be useful as well (FIGS. 2A-2B). This, for example, may be used to reduce the rate at which magnetic beads are pelleted in order to reduce the shearing forces experienced between the pelleting beads and the liquid supernatant (since shear force is proportional to velocity). This modulation of the magnetic field strength may be accomplished in a few different ways. In one embodiment, a permanent magnet may be moved orthogonally with respect to the EWOD surface in order to increase or decrease the magnetic field strength in the vicinity of the magnetic beads. In an alternative embodiment, an electromagnet or electro-permanent magnet may be used instead of a permanent magnet in order to electronically modulate the strength of the magnetic field in a time-dependent manner.

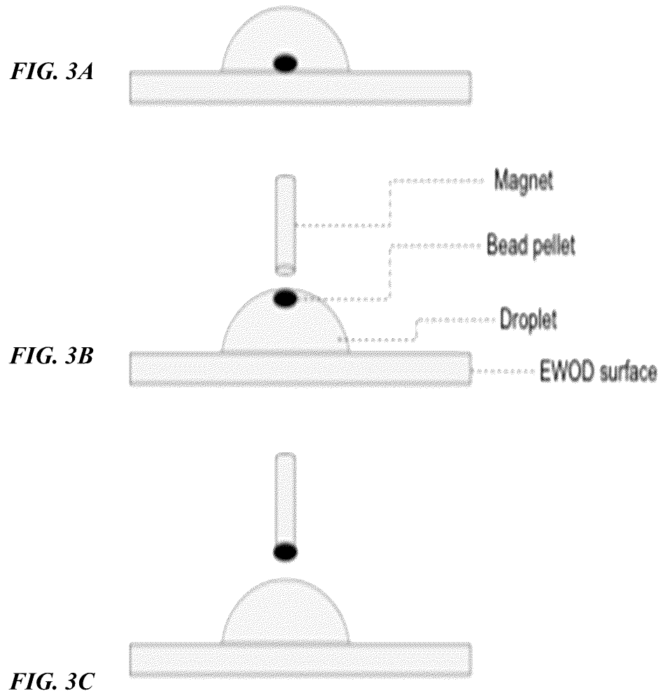

Small (<40 μL) and/or high surface tension droplets can also pose a problem for splitting the bead pellet from the supernatant. Separating droplets where the magnetic beads take up a relatively significant volume relative to the overall bead and droplet volume can also pose this problem. In these cases, it may be beneficial to use gravity and surface tension as additional forces to aid in the separation. This may be accomplished, for example, by introducing a magnet above the droplet and bead pellet (FIGS. 3A-3C). This magnet may be a permanent magnet, electromagnet, or electro-permanent magnet. The magnet would be positioned vertically above the droplet in such a way that the bead pellet would be pulled upward out of the droplet. The position and strength of the magnet could be tuned such that it results in a clean separation between the bead pellet and the droplet supernatant.

When a magnet is used for magnetic bead manipulation on a surface, it may be beneficial to move the magnet away in a controlled fashion for various reason. In this method, the magnet is moved orthogonally to the active surface so that there is enough clearance between the magnet and the bottom side of the fluidic manipulation device. A similar effect may be accomplished by moving the magnet parallel to the surface but in a manner such that the magnet is completely outside the perimeter of the active surface and provides sufficient clearance below the surface.

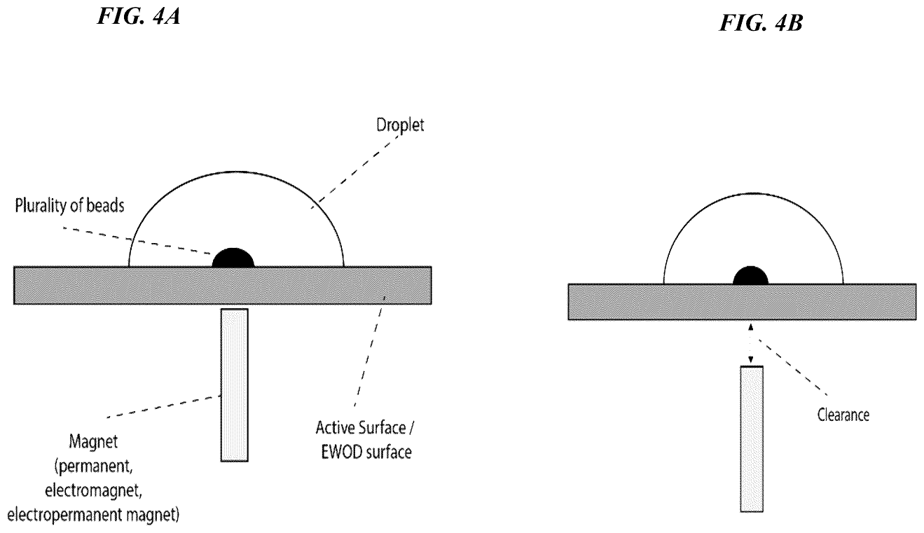

One magnet position of this method is depicted in FIG. 4A. The magnet is up close to the active surface of the fluidic manipulation device. Another embodiment of the magnet position of this method is depicted in FIG. 4B. The magnet has moved orthogonally to provide clearance between the magnet and surface. Another embodiment of the magnet position of this method is depicted in FIG. 4C. The active surface moves up and down to create agitation in the droplet surrounding the magnetic bead pellet. In the embodiments of FIGS. 4B-4C, while the magnetic field strength may have reduced, it is still strong enough to keep the magnetic bead pellet in place while the agitation step continues. Additionally, the agitation step can be replaced with another droplet operation. Droplet operation can include droplet being stationary, droplet moving back and forth on the beads using electrowetting, droplet being heated or cooled, a small aliquot from the droplet being removed, droplet splitting, droplet dispense, or any combinations thereof.

Creating this clearance and introducing the droplet operation while the beads are held in place functions as an aide to electrowetting forces when the forces are not enough to manipulate the droplet. Droplet operation while magnet is still below the surface allows for improved mobility of droplets and/or improved droplet operations.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a”, “an” and “the” include plural references unless the context clearly dictates otherwise. For example, the term “a sample” includes a plurality of samples, including combinations thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values, can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

The term “droplet”, as used herein, generally refers to a discrete or finite volume of a fluid (e.g., a liquid). A droplet may be generated by one phase separated from another phase by an interface. The droplet may be a first phase phase-separated from another phase. The droplet me include a single phase or multiple phases (e.g., an aqueous phase containing a polymer or an emulsion). The droplet may be a liquid phase disposed adjacent to a surface and in contact with a separate phase (e.g., gas phase, such as air).

The term “biological sample,” as used herein, generally refers to a biological material. Such biological material may display bioactivity or be bioactive. Such biological material may be, or may include, a deoxyribonucleic acid (DNA) molecule, a ribonucleic acid (R A) molecule, a polypeptide (e.g., protein), or any combination thereof. A biological sample (or sample) may be a tissue sample, such as a biopsy, core biopsy, needle aspirate, or fine needle aspirate. The sample may be a fluid sample, such as a blood sample, urine sample, stool sample, or saliva sample. The sample may be a skin sample. The sample may be a cheek swab. The sample may be a plasma or serum sample. The sample may be a plant derived sample, water sample or soil sample. The sample may be extraterrestrial. The extraterrestrial sample may contain biological material. The sample may be a cell-free (or cell free) sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears. The sample may include a eukaryotic cell or a plurality thereof. The sample may include a prokaryotic cell or a plurality thereof. The sample may include a virus. The sample may include a compound derived from an organism. The sample may be from a plant. The sample may be from an animal. The sample may be from an animal suspected of having or carrying a disease. The sample may be from a mammal.

The term “electro-mechanical actuator,” as used herein, generally refers to a non-human structure that can be utilized to apply vibration and/or acoustic forces to the arrays described herein. By way of non-limiting examples, electro-mechanical actuators include an oscillating mechanism or cantilever, motor-driven linkages, and/or rotating masses. In some embodiments, the electro-mechanical actuators described herein are flexible structures comprising various flexible elements (e.g. a linear flexure) or with traditional bearings.

The term “electrowetting,” as used herein, generally refers to any liquid handling technology which uses voltage applied to electrodes or other conductors to move fluids on a surface. The surface tension and wetting properties of a fluid may be altered by electric fields using the electrowetting effect. The electrowetting effect may arise from the change in solid-liquid contact angle due to an applied potential difference between the solid and the liquid. When the fluid is provided as a droplet, differences in wetting surface tension may vary over the width of the droplet, and corresponding change in contact angle, may provide motive force to cause the droplet to move, without moving parts or physical contact.

The section headings used herein are for organizational purposes only and are not to be construed as limiting the subject matter described.

### Electrowetting Devices and Systems

An electrowetting device may be used to move individual droplets of water (or other aqueous, polar, or conducting solution) from place to place. The surface tension and wetting properties of water may be altered by electric field strength using the electrowetting effect. The electrowetting effect may arise from the change in solid-liquid contact angle due to an applied potential difference between the solid and the liquid. Differences in wetting surface tension that may vary over the width of the droplet, and corresponding change in contact angle, may provide motive force to cause the droplets to move, without moving parts or physical contact. The electrowetting device may include a grid of electrodes with a dielectric layer with appropriate electrical and surface priorities overlaying electrodes, all laid on a rigid insulating substrate. Additional examples of electrowetting devices can be found in WO2021041709, which is hereby incorporated by reference in its entirety.

The surface of the electrode grid may be prepared so that it has low adhesion with water. This may allow water droplets to be moved along the surface by small forces generated by gradients in electric field and surface tension across the width of the droplet. A surface with low adhesion may reduce the trail left behind from a droplet. A smaller trail may reduce droplet cross contamination, and may reduce sample loss during droplet movement. Low adhesion to surface may also allow for low actuation voltage for droplet motion and repeatable behavior of droplet motion. There are several ways to measure low adhesion between a surface and a droplet including slide angle and contact angle hysteresis, such as, for example, using a contact angle goniometer or a charge-coupled device (CCD) camera.

There may be several ways to achieve low surface adhesion; for example, mechanically polishing, chemically etching, or a combination thereof until smooth within a few nanometers, applying coating to fill surface irregularities, applying liquids to fill surface irregularities, chemically modifying the surface to create desirable surface properties (hydrophobic, hydrophilic, resistance to biofouling, varying with electric field strength, etc.).

### Electrowetting on a Dielectric (EWOD) for Droplet Manipulation

In some embodiments, Electrowetting on Dielectric (EWOD) is a phenomenon in which the wettability of an aqueous, polar, or conducting liquid (L) may be modulated through an electric field across a dielectric film between the droplet and conducting electrode. Adding or subtracting charge from electrode may change the wettability of an insulating dielectric layer, and that wettability change is reflected in a change to contact angle of the droplet. The contact angle change may in turn cause the droplet to change shape, to move, to split into smaller droplets, or to merge with another droplet. Additional examples EWOD droplet actuation can be found in WO2021041709, which is hereby incorporated by reference in its entirety.

### Droplet Motion, Merging and Splitting

A droplet may be moved, merged, split, or any combination thereof on an open surface electrowetting device. The same principles apply to two plate configuration (droplet sandwiched).

In some embodiments, applying a voltage to an electrode may make the overlying surface hydrophilic and a droplet can then wet it. When voltage is applied on two neighboring electrodes, the droplet may spread across both actuated electrodes. When voltage is removed from electrode and applied to another adjacent electrode, the surface returns to original hydrophobic state and the droplet may be pushed out. By sequentially controlling the voltage applied to an electrode grid, a droplet's position on a surface may be precisely controlled.

In some embodiments, when two droplets are pulled towards the same electrode, they may naturally merge due to surface tension. This principle may be applied to merge a number of droplets to create a larger volume droplet spreading across multiple electrodes.

In some embodiments, a droplet may be split into two smaller ones through a sequence of voltages, applied across multiple electrodes (at least three electrodes). In some embodiments, a single large droplet is consolidated above a single electrode. In some embodiments, an equal voltage is applied to three adjacent electrodes simultaneously, and this may cause the single droplet to spread across the three adjacent electrodes. In some embodiments, turning off the center electrode may force the droplet to move out to the two outer electrodes. Due to the equal potential on both of the two outer electrodes, the droplet may then split into two smaller droplets.

### EWOD-Enabled Magnetic Bead Wash

Magnetic particles may be manipulated on the surface of the chip by a controllable, localized magnetic field. The magnetic particles may be made of, for example, microspheres. Controlling the localized magnetic field may be achieved by, for example, placing a solenoid, a magnet, a pair of magnets, or any combination thereof in the vicinity of the particles or by generating a magnetic field within the EWOD chip. Magnetic bead-based separations and washes may be performed on an EWOD-enabled array. The droplet may be manipulated using the actuating electrodes which may also allow positioning of the droplet. The magnetic particles may be concentrated in a small region using the magnetic field. Liquids may be separated from the magnetic particles by EWOD-based, dielectrophoresis-based, or other electromotive force based actuation. Separation is possible in the open-plate and two-plate systems. Since the droplet can be positioned using EWOD actuation, the fluid may also be aspirated from the chip using a liquid handling robot, leaving the magnetic particles on the chip surface. Removal of liquid may be achieved through a hole, or a plurality thereof, in the array by employing capillary forces, pneumatic forces, electromotive forces, such as EWOD or dielectrowetting, or any combination thereof. This waste fluid may be collected in a reservoir positioned under the array. A computer-vision-based algorithm may be used to inform and provide feedback to the liquid handler and/or array for the processes involving magnetic beads. The processes may include, for example, aspiration of the supernatant, resuspension of beads, preventing aspiration of magnetic beads along with the supernatant during removal of supernatant, or any combination thereof.

### Vibration-Assisted Mixing

Liquid droplets can be mixed in a variety of methods. The present disclosure provides methods by which vibration of a digital microfluidic surface can be used to assist in the mixing of liquids on the surface of the digital microfluidic device. The vibration may produce small-scale fluidic motion within a droplet on the surface of the digital microfluidic device. The motion may encourage diffusion and rapidly speed up the mixing process. An example of the benefits of vibration-assisted droplet mixing is efficient capture of the DNA onto the magnetic microparticles (e.g. beads) and ultimately higher yield DNA extraction. In some embodiments, an electrowetting array comprising an open surface is provided.

A common problem with digital microfluidics platforms is achieving robust mixing with all varieties of reagents and droplets. Highly viscous liquid droplets, for example, can be extremely difficult to mix effectively using a purely electrowetting based motion. These kinds of viscous droplets are important in a wide range of applications including DNA extraction from highly concentrated sample material where DNA needs to be efficiently bound to magnetic beads. Using purely electrowetting based motion to mix in these applications results in very poor mixing and therefore very poor DNA extraction from the sample droplet.

Implementation of devices, systems, and methods by which vibration and/or application of acoustic forces to the digital microfluidic surface can be used to assist in the mixing of liquids on the surface is described herein. The vibration, when tuned to the appropriate frequency and amplitude, produces small-scale fluidic motion within the droplet that encourages diffusion and rapidly speeds up the mixing process.

Vibration also contributes to enhanced mobility of droplets. This is especially true for droplets that contain particulates. Without vibration, large particles can tend to settle at the interface between the droplet and the substrate. When these particles are present at the droplet's contact line they can act to pin the droplet in place, restricting its mobility. The introduction of vibration can help keep particles from settling at the contact line and, in doing so, greatly improves the reliability of electrowetting mobility of particulate-carrying droplets.

Vibration based mixing is synergistic with electrowetting based mixing. While vibration mixing is effective at dispersing particles within portions of a liquid droplet, it is often less effective at macro-scale mixing across the entire droplet, especially for droplets with low contact angle with the surface. Electrowetting-based droplet mixing helps address this problem and with both vibration and electrowetting acting together, mixing of a wide variety of droplets of various compositions can be accomplished rapidly and effectively.

An aspect of the present disclosure comprises a method of processing a droplet comprising providing an electrowetting array, wherein said electrowetting array is adjacent to a magnet configured to apply a magnetic field to said electrowetting array in one or more of three directional axes, providing on said array a droplet comprising one or more artifacts that are responsive to said magnetic field and actuating said magnet in said one or more of three directional axes with respect to said array to separate said one or more artifacts from said droplet.

In some embodiments, said magnet is moved parallel to said array in the x direction. In some embodiments, said magnet is moved parallel to said array in the y direction. In some embodiments, said magnet is moved orthogonally to said array.

In some embodiments, the strength of said magnetic field is modulated. In some embodiments, the strength of said magnetic field is increased. In some embodiments, the strength of said magnetic field is decreased. In some embodiments, the strength of said magnetic field is not modulated. In some embodiments, the strength of said magnetic field remains essentially the same. In some embodiments, the strength of said magnetic field remains the same.

In some embodiments, said magnet is a permanent magnet. In some embodiments, said magnet is an electromagnet or an electro-permanent magnet. In some embodiments, said electromagnet or said electro-permanent magnet modulates the strength of said magnetic field in a time-dependent manner.

In some embodiments, said magnet is positioned above said electrowetting array. In some embodiments, said magnet moves orthogonally with respect to said electrowetting array to separate said one or more artifacts from said droplet. In some embodiments, said magnet is positioned below said electrowetting array.

An aspect of the present disclosure comprises a method of removing one or more artifacts from a droplet comprising providing a magnet configured to apply a magnetic field to said droplet and actuating said magnet with respect to said droplet to separate said one or more artifacts from said droplet, wherein said droplet is less than 40 microliters.

In some embodiments, said magnet is moved parallel to said array in the x direction. In some embodiments, said magnet is moved parallel to said array in the y direction. In some embodiments, said magnet is moved orthogonally to said array.

In some embodiments, the strength of said magnetic field is modulated. In some embodiments, the strength of said magnetic field is increased. In some embodiments, the strength of said magnetic field is decreased.

In some embodiments, said magnet is a permanent magnet. In some embodiments, said magnet is an electromagnet or an electro-permanent magnet. In some embodiments, said electromagnet or said electro-permanent magnet modulates the strength of said magnetic field in a time-dependent manner.

In some embodiments, said magnet is positioned above said electrowetting array. In some embodiments, said magnet moves orthogonally with respect to said electrowetting array to separate said one or more artifacts from said droplet. In some embodiments, said magnet is positioned below said electrowetting array.

In some embodiments, said droplet is less than 30 microliters. In some embodiments, said droplet is less than 20 microliters. In some embodiments, said droplet is less than 10 microliters.

Another aspect of the instant disclosure is a method for processing a droplet, the method comprising: providing the droplet on a surface, wherein the surface is adjacent to a magnet configured to provide a magnetic field contacting the droplet, and wherein the droplet comprises one or more artifacts that are responsive to the magnetic field, displacing the magnet proximal to the droplet, wherein the magnetic field contacting the droplet comprises a flux density of at least about 4 millitesla (“mT”) to at least about 10 mT, thereby manipulating the one or more artifacts that are responsive to the magnetic field. In some embodiments, the magnet is displaced along an axis orthogonal to the surface. In some embodiments, the magnet is displaced at a distance of about 0 millimeters to about 15 millimeters from the surface. In some embodiments, the surface comprises an electrowetting array. In some embodiments, the one or more artifacts that are responsive to the magnetic field comprise a nucleic acid molecule. In some embodiments, the one or more artifacts that are responsive to the magnetic field comprise a plurality of nucleic acid molecules. In some embodiments, the magnet is displaced along an axis parallel to the surface. In some embodiments, the method further comprises motioning the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters.

In some embodiments, the nucleic acid molecule comprises at least 100 kb. In some embodiments, the nucleic acid molecule comprises at least 10 kb to 200 kb. In some embodiments, the nucleic acid molecule comprises at least 10 kb to 20 kb, 10 kb to 40 kb, 10 kb to 50 kb, 10 kb to 70 kb, 10 kb to 90 kb, 10 kb to 110 kb, 10 kb to 130 kb, 10 kb to 150 kb, 10 kb to 170 kb, 10 kb to 190 kb, 10 kb to 200 kb, 20 kb to 40 kb, 20 kb to 50 kb, 20 kb to 70 kb, 20 kb to 90 kb, 20 kb to 110 kb, 20 kb to 130 kb, 20 kb to 150 kb, 20 kb to 170 kb, 20 kb to 190 kb, 20 kb to 200 kb, 40 kb to 50 kb, 40 kb to 70 kb, 40 kb to 90 kb, 40 kb to 110 kb, 40 kb to 130 kb, 40 kb to 150 kb, 40 kb to 170 kb, 40 kb to 190 kb, 40 kb to 200 kb, 50 kb to 70 kb, 50 kb to 90 kb, 50 kb to 110 kb, 50 kb to 130 kb, 50 kb to 150 kb, 50 kb to 170 kb, 50 kb to 190 kb, 50 kb to 200 kb, 70 kb to 90 kb, 70 kb to 110 kb, 70 kb to 130 kb, 70 kb to 150 kb, 70 kb to 170 kb, 70 kb to 190 kb, 70 kb to 200 kb, 90 kb to 110 kb, 90 kb to 130 kb, 90 kb to 150 kb, 90 kb to 170 kb, 90 kb to 190 kb, 90 kb to 200 kb, 110 kb to 130 kb, 110 kb to 150 kb, 110 kb to 170 kb, 110 kb to 190 kb, 110 kb to 200 kb, 130 kb to 150 kb, 130 kb to 170 kb, 130 kb to 190 kb, 130 kb to 200 kb, 150 kb to 170 kb, 150 kb to 190 kb, 150 kb to 200 kb, 170 kb to 190 kb, 170 kb to 200 kb, or 190 kb to 200 kb. In some embodiments, the nucleic acid molecule comprises at least 10 kb, 20 kb, 40 kb, 50 kb, 70 kb, 90 kb, 110 kb, 130 kb, 150 kb, 170 kb, 190 kb, or 200 kb. In some embodiments, the nucleic acid molecule comprises at least 10 kb, 20 kb, 40 kb, 50 kb, 70 kb, 90 kb, 110 kb, 130 kb, 150 kb, 170 kb, or 190 kb. In some embodiments, the nucleic acid molecule comprises at least 200 kb to 1,000 kb. In some embodiments, the nucleic acid molecule comprises at least 200 kb to 300 kb, 200 kb to 400 kb, 200 kb to 500 kb, 200 kb to 600 kb, 200 kb to 700 kb, 200 kb to 800 kb, 200 kb to 900 kb, 200 kb to 1,000 kb, 300 kb to 400 kb, 300 kb to 500 kb, 300 kb to 600 kb, 300 kb to 700 kb, 300 kb to 800 kb, 300 kb to 900 kb, 300 kb to 1,000 kb, 400 kb to 500 kb, 400 kb to 600 kb, 400 kb to 700 kb, 400 kb to 800 kb, 400 kb to 900 kb, 400 kb to 1,000 kb, 500 kb to 600 kb, 500 kb to 700 kb, 500 kb to 800 kb, 500 kb to 900 kb, 500 kb to 1,000 kb, 600 kb to 700 kb, 600 kb to 800 kb, 600 kb to 900 kb, 600 kb to 1,000 kb, 700 kb to 800 kb, 700 kb to 900 kb, 700 kb to 1,000 kb, 800 kb to 900 kb, 800 kb to 1,000 kb, or 900 kb to 1,000 kb. In some embodiments, the nucleic acid molecule comprises at least 200 kb, 300 kb, 400 kb, 500 kb, 600 kb, 700 kb, 800 kb, 900 kb, or 1,000 kb. In some embodiments, the nucleic acid molecule comprises at least at least 200 kb, 300 kb, 400 kb, 500 kb, 600 kb, 700 kb, 800 kb, or 900 kb. In some embodiments, the nucleic acid molecule comprises at least at most 300 kb, 400 kb, 500 kb, 600 kb, 700 kb, 800 kb, 900 kb, or 1,000 kb.

In some embodiments, the nucleic acid molecule comprises at least 10 kb to 1,000 kb. In some embodiments, the nucleic acid molecule comprises at least 10 kb to 20 kb, 10 kb to 50 kb, 10 kb to 75 kb, 10 kb to 100 kb, 10 kb to 200 kb, 10 kb to 300 kb, 10 kb to 400 kb, 10 kb to 500 kb, 10 kb to 750 kb, 10 kb to 900 kb, 10 kb to 1,000 kb, 20 kb to 50 kb, 20 kb to 75 kb, 20 kb to 100 kb, 20 kb to 200 kb, 20 kb to 300 kb, 20 kb to 400 kb, 20 kb to 500 kb, 20 kb to 750 kb, 20 kb to 900 kb, 20 kb to 1,000 kb, 50 kb to 75 kb, 50 kb to 100 kb, 50 kb to 200 kb, 50 kb to 300 kb, 50 kb to 400 kb, 50 kb to 500 kb, 50 kb to 750 kb, 50 kb to 900 kb, 50 kb to 1,000 kb, 75 kb to 100 kb, 75 kb to 200 kb, 75 kb to 300 kb, 75 kb to 400 kb, 75 kb to 500 kb, 75 kb to 750 kb, 75 kb to 900 kb, 75 kb to 1,000 kb, 100 kb to 200 kb, 100 kb to 300 kb, 100 kb to 400 kb, 100 kb to 500 kb, 100 kb to 750 kb, 100 kb to 900 kb, 100 kb to 1,000 kb, 200 kb to 300 kb, 200 kb to 400 kb, 200 kb to 500 kb, 200 kb to 750 kb, 200 kb to 900 kb, 200 kb to 1,000 kb, 300 kb to 400 kb, 300 kb to 500 kb, 300 kb to 750 kb, 300 kb to 900 kb, 300 kb to 1,000 kb, 400 kb to 500 kb, 400 kb to 750 kb, 400 kb to 900 kb, 400 kb to 1,000 kb, 500 kb to 750 kb, 500 kb to 900 kb, 500 kb to 1,000 kb, 750 kb to 900 kb, 750 kb to 1,000 kb, or 900 kb to 1,000 kb. In some embodiments, the nucleic acid molecule comprises at least 10 kb, 20 kb, 50 kb, 75 kb, 100 kb, 200 kb, 300 kb, 400 kb, 500 kb, 750 kb, 900 kb, or 1,000 kb. In some embodiments, the nucleic acid molecule comprises at least 10 kb, 20 kb, 50 kb, 75 kb, 100 kb, 200 kb, 300 kb, 400 kb, 500 kb, 750 kb, or 900 kb.

In some embodiments, at least 20% of the plurality of nucleic acid molecules comprise at least 100 kb. In some embodiments, at least 30% of the plurality of nucleic acid molecules comprise at least 100 kb. In some embodiments, at least 40% of the plurality of nucleic acid molecules comprise at least 100 kb. In some embodiments, at least 50% of the plurality of nucleic acid molecules comprise at least 100 kb. In some embodiments, at least 60% of the plurality of nucleic acid molecules comprise at least 100 kb. In some embodiments, at least 70% of the plurality of nucleic acid molecules comprise at least 100 kb. In some embodiments, at least 80% of the plurality of nucleic acid molecules comprise at least 100 kb. In some embodiments, at least 90% of the plurality of nucleic acid molecules comprise at least 100 kb.

In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the method further comprises motioning the magnet and maintaining at least 30% the plurality of nucleic acid molecules at 100 kb or more. In some embodiments, the method further comprises motioning the magnet and maintaining at least 40% the plurality of nucleic acid molecules at 100 kb or more. In some embodiments, the method further comprises motioning the magnet and maintaining at least 50% the plurality of nucleic acid molecules at 100 kb or more. In some embodiments, the method further comprises motioning the magnet and maintaining at least 60% the plurality of nucleic acid molecules at 100 kb or more. In some embodiments, the method further comprises motioning the magnet and maintaining at least 70% the plurality of nucleic acid molecules at 100 kb or more. In some embodiments, the method further comprises motioning the magnet and maintaining at least 80% the plurality of nucleic acid molecules at 100 kb or more. In some embodiments, the method further comprises motioning the magnet and maintaining at least 90% the plurality of nucleic acid molecules at 100 kb or more.

In some embodiments, the flux density is sufficient for immobilizing the one or more artifacts that are responsive to the magnetic field during one or more droplet operations. In some embodiments, the one or more droplet operations comprise agitation of the surface. In some embodiments, the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the one or more droplet operations comprise the droplet being stationary. In some embodiments, the one or more droplet operations comprise the droplet moving back and forth on the beads using electrowetting. In some embodiments, the one or more droplet operations comprise the droplet being heated or cooled. In some embodiments, the one or more droplet operations comprise a small aliquot from the droplet being removed. In some embodiments, the one or more droplet operations comprise droplet splitting. In some embodiments, the one or more droplet operations comprise the droplet being dispensed.

Another aspect of the instant disclosure is a method for processing a droplet, the method comprising: providing the droplet on a surface, wherein the surface is adjacent to a magnet configured to provide a magnetic field contacting the droplet, and wherein the droplet comprises one or more artifacts that are responsive to the magnetic field; displacing the magnet proximal to the droplet at a distance of about 0 millimeters to about 15 millimeters from the surface, thereby manipulating the one or more artifacts that are responsive to the magnetic field. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a nucleic acid molecule. In some embodiments, the one or more artifacts that are responsive to the magnetic field comprise a plurality of nucleic acid molecules. In some embodiments, the magnet is displaced along an axis parallel to the surface. In some embodiments, the method further comprises motion the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters.

In some embodiments, the nucleic acid molecule comprises at least 100 kb. In some embodiments, the nucleic acid molecule comprises at least 100 kb to 1,000 kb. In some embodiments, the nucleic acid molecule comprises at least 100 kb to 200 kb, 100 kb to 300 kb, 100 kb to 400 kb, 100 kb to 500 kb, 100 kb to 600 kb, 100 kb to 700 kb, 100 kb to 800 kb, 100 kb to 900 kb, 100 kb to 1,000 kb, 200 kb to 300 kb, 200 kb to 400 kb, 200 kb to 500 kb, 200 kb to 600 kb, 200 kb to 700 kb, 200 kb to 800 kb, 200 kb to 900 kb, 200 kb to 1,000 kb, 300 kb to 400 kb, 300 kb to 500 kb, 300 kb to 600 kb, 300 kb to 700 kb, 300 kb to 800 kb, 300 kb to 900 kb, 300 kb to 1,000 kb, 400 kb to 500 kb, 400 kb to 600 kb, 400 kb to 700 kb, 400 kb to 800 kb, 400 kb to 900 kb, 400 kb to 1,000 kb, 500 kb to 600 kb, 500 kb to 700 kb, 500 kb to 800 kb, 500 kb to 900 kb, 500 kb to 1,000 kb, 600 kb to 700 kb, 600 kb to 800 kb, 600 kb to 900 kb, 600 kb to 1,000 kb, 700 kb to 800 kb, 700 kb to 900 kb, 700 kb to 1,000 kb, 800 kb to 900 kb, 800 kb to 1,000 kb, or 900 kb to 1,000 kb. In some embodiments, the nucleic acid molecule comprises at least 100 kb, 200 kb, 300 kb, 400 kb, 500 kb, 600 kb, 700 kb, 800 kb, 900 kb, or 1,000 kb. In some embodiments, the nucleic acid molecule comprises at least 100 kb, 200 kb, 300 kb, 400 kb, 500 kb, 600 kb, 700 kb, 800 kb, or 900 kb.

In some embodiments, the nucleic acid molecule comprises at least 10 kb to 100 kb. In some embodiments, the nucleic acid molecule comprises at least 10 kb to 20 kb, 10 kb to 50 kb, 10 kb to 75 kb, 10 kb to 90 kb, 10 kb to 100 kb, 20 kb to 50 kb, 20 kb to 75 kb, 20 kb to 90 kb, 20 kb to 100 kb, 50 kb to 75 kb, 50 kb to 90 kb, 50 kb to 100 kb, 75 kb to 90 kb, 75 kb to 100 kb, or 90 kb to 100 kb. In some embodiments, the nucleic acid molecule comprises at least 10 kb, 20 kb, 50 kb, 75 kb, 90 kb, or 100 kb. In some embodiments, the nucleic acid molecule comprises at least 10 kb, 20 kb, 50 kb, 75 kb, or 90 kb.

In some embodiments, the nucleic acid molecule comprises at least 10 kb to 1,000 kb. In some embodiments, the nucleic acid molecule comprises at least 10 kb to 20 kb, 10 kb to 50 kb, 10 kb to 75 kb, 10 kb to 100 kb, 10 kb to 200 kb, 10 kb to 300 kb, 10 kb to 400 kb, 10 kb to 500 kb, 10 kb to 750 kb, 10 kb to 900 kb, 10 kb to 1,000 kb, 20 kb to 50 kb, 20 kb to 75 kb, 20 kb to 100 kb, 20 kb to 200 kb, 20 kb to 300 kb, 20 kb to 400 kb, 20 kb to 500 kb, 20 kb to 750 kb, 20 kb to 900 kb, 20 kb to 1,000 kb, 50 kb to 75 kb, 50 kb to 100 kb, 50 kb to 200 kb, 50 kb to 300 kb, 50 kb to 400 kb, 50 kb to 500 kb, 50 kb to 750 kb, 50 kb to 900 kb, 50 kb to 1,000 kb, 75 kb to 100 kb, 75 kb to 200 kb, 75 kb to 300 kb, 75 kb to 400 kb, 75 kb to 500 kb, 75 kb to 750 kb, 75 kb to 900 kb, 75 kb to 1,000 kb, 100 kb to 200 kb, 100 kb to 300 kb, 100 kb to 400 kb, 100 kb to 500 kb, 100 kb to 750 kb, 100 kb to 900 kb, 100 kb to 1,000 kb, 200 kb to 300 kb, 200 kb to 400 kb, 200 kb to 500 kb, 200 kb to 750 kb, 200 kb to 900 kb, 200 kb to 1,000 kb, 300 kb to 400 kb, 300 kb to 500 kb, 300 kb to 750 kb, 300 kb to 900 kb, 300 kb to 1,000 kb, 400 kb to 500 kb, 400 kb to 750 kb, 400 kb to 900 kb, 400 kb to 1,000 kb, 500 kb to 750 kb, 500 kb to 900 kb, 500 kb to 1,000 kb, 750 kb to 900 kb, 750 kb to 1,000 kb, or 900 kb to 1,000 kb. In some embodiments, the nucleic acid molecule comprises at least 10 kb, 20 kb, 50 kb, 75 kb, 100 kb, 200 kb, 300 kb, 400 kb, 500 kb, 750 kb, 900 kb, or 1,000 kb. In some embodiments, the nucleic acid molecule comprises at least 10 kb, 20 kb, 50 kb, 75 kb, 100 kb, 200 kb, 300 kb, 400 kb, 500 kb, 750 kb, or 900 kb.

In some embodiments, at least 20% of the plurality of nucleic acid molecules comprise at least 100 kb. In some embodiments, at least 30% of the plurality of nucleic acid molecules comprise at least 100 kb. In some embodiments, at least 40% of the plurality of nucleic acid molecules comprise at least 100 kb. In some embodiments, at least 50% of the plurality of nucleic acid molecules comprise at least 100 kb. In some embodiments, at least 60% of the plurality of nucleic acid molecules comprise at least 100 kb. In some embodiments, at least 70% of the plurality of nucleic acid molecules comprise at least 100 kb. In some embodiments, at least 80% of the plurality of nucleic acid molecules comprise at least 100 kb. In some embodiments, at least 90% of the plurality of nucleic acid molecules comprise at least 100 kb.

In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the method further comprises motioning the magnet and maintaining at least 30% the plurality of nucleic acid molecules at 100 kb or more. In some embodiments, the method further comprises motioning the magnet and maintaining at least 40% the plurality of nucleic acid molecules at 100 kb or more. In some embodiments, the method further comprises motioning the magnet and maintaining at least 50% the plurality of nucleic acid molecules at 100 kb or more. In some embodiments, the method further comprises motioning the magnet and maintaining at least 60% the plurality of nucleic acid molecules at 100 kb or more. In some embodiments, the method further comprises motioning the magnet and maintaining at least 70% the plurality of nucleic acid molecules at 100 kb or more. In some embodiments, the method further comprises motioning the magnet and maintaining at least 80% the plurality of nucleic acid molecules at 100 kb or more. In some embodiments, the method further comprises motioning the magnet and maintaining at least 90% the plurality of nucleic acid molecules at 100 kb or more.

In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the distance is sufficient for immobilizing the one or more artifacts that are responsive to the magnetic field during one or more droplet operations. In some embodiments, the one or more droplet operations comprise agitation of the surface. In some embodiments, the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the one or more droplet operations comprise the droplet being stationary. In some embodiments, the one or more droplet operations comprise the droplet moving back and forth on the beads using electrowetting. In some embodiments, the one or more droplet operations comprise the droplet being heated or cooled. In some embodiments, the one or more droplet operations comprise a small aliquot from the droplet being removed. In some embodiments, the one or more droplet operations comprise droplet splitting. In some embodiments, the one or more droplet operations comprise the droplet being dispensed.

Another aspect of the instant disclosure is a method for processing a droplet, the method comprising: providing the droplet on a surface, wherein the surface is adjacent to a magnet configured to provide a magnetic field contacting the droplet, wherein the droplet comprises one or more artifacts that are responsive to the magnetic field, and wherein the one or more artifacts that are responsive to the magnetic field comprise a nucleic acid molecule; motioning the magnet and maintaining the nucleic acid molecule at 100 kb or more, thereby manipulating the one or more artifacts that are responsive to the magnetic field. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the method further comprises motion the magnet and maintaining the flux density of at least about 4 millitesla (“mT”) to at least about 10 mT of the magnetic field contacting the droplet. In some embodiments, the method further comprises motioning the magnet and maintaining the distance of about 0 millimeters to about 15 millimeters from the surface. In some embodiments, the droplet is less than 30 microliters. In some embodiments, the droplet is less than 20 microliters. In some embodiments, the droplet is less than 10 microliters.

In some embodiments, at least 20% of the plurality of nucleic acid molecules comprise at least 100 kb. In some embodiments, at least 30% of the plurality of nucleic acid molecules comprise at least 100 kb. In some embodiments, at least 40% of the plurality of nucleic acid molecules comprise at least 100 kb. In some embodiments, at least 50% of the plurality of nucleic acid molecules comprise at least 100 kb. In some embodiments, at least 60% of the plurality of nucleic acid molecules comprise at least 100 kb. In some embodiments, at least 70% of the plurality of nucleic acid molecules comprise at least 100 kb. In some embodiments, at least 80% of the plurality of nucleic acid molecules comprise at least 100 kb. In some embodiments, at least 90% of the plurality of nucleic acid molecules comprise at least 100 kb.

In some embodiments, the method further comprises maintaining at least 20% the plurality of nucleic acid molecules at 100 kb or more. In some embodiments, the method further comprises maintaining at least 30% the plurality of nucleic acid molecules at 100 kb or more. In some embodiments, the method further comprises maintaining at least 40% the plurality of nucleic acid molecules at 100 kb or more. In some embodiments, the method further comprises maintaining at least 50% the plurality of nucleic acid molecules at 100 kb or more. In some embodiments, the method further comprises maintaining at least 60% the plurality of nucleic acid molecules at 100 kb or more. In some embodiments, the method further comprises maintaining at least 70% the plurality of nucleic acid molecules at 100 kb or more. In some embodiments, the method further comprises maintaining at least 80% the plurality of nucleic acid molecules at 100 kb or more. In some embodiments, the method further comprises maintaining at least 90% the plurality of nucleic acid molecules at 100 kb or more.

In some embodiments, the method further comprises motioning the magnet and maintaining the nucleic acid molecule at 100 kb or more. In some embodiments, the method further comprises motioning the magnet and maintaining at least 20% the plurality of nucleic acid molecules at 100 kb or more. In some embodiments, the method further comprises motioning the magnet and maintaining at least 30% the plurality of nucleic acid molecules at 100 kb or more. In some embodiments, the method further comprises motioning the magnet and maintaining at least 40% the plurality of nucleic acid molecules at 100 kb or more. In some embodiments, the method further comprises motioning the magnet and maintaining at least 50% the plurality of nucleic acid molecules at 100 kb or more. In some embodiments, the method further comprises motioning the magnet and maintaining at least 60% the plurality of nucleic acid molecules at 100 kb or more. In some embodiments, the method further comprises motioning the magnet and maintaining at least 70% the plurality of nucleic acid molecules at 100 kb or more. In some embodiments, the method further comprises motioning the magnet and maintaining at least 80% the plurality of nucleic acid molecules at 100 kb or more. In some embodiments, the method further comprises motioning the magnet and maintaining at least 90% the plurality of nucleic acid molecules at 100 kb or more.

In some embodiments, the distance is sufficient for immobilizing the one or more artifacts that are responsive to the magnetic field during one or more droplet operations. In some embodiments, the one or more droplet operations comprise agitation of the surface. In some embodiments, the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the one or more droplet operations comprise the droplet being stationary. In some embodiments, the one or more droplet operations comprise the droplet moving back and forth on the beads using electrowetting. In some embodiments, the one or more droplet operations comprise the droplet being heated or cooled. In some embodiments, the one or more droplet operations comprise a small aliquot from the droplet being removed. In some embodiments, the one or more droplet operations comprise droplet splitting. In some embodiments, the one or more droplet operations comprise the droplet being dispensed.

Another aspect of the instant disclosure is a system for processing a droplet, the system comprising: a surface configured to support a droplet; a magnet adjacent the surface, wherein the magnet is configured to provide a magnetic field contacting the droplet, wherein the droplet comprises one or more artifacts that are responsive to the magnetic field, and wherein the one or more artifacts that are responsive to the magnetic field comprise a nucleic acid molecule; a controller mechanically coupled to the magnet, wherein the controller is configured to displace the magnet proximate to the droplet and maintaining the nucleic acid molecule at 100 kb or more. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the controller is configured maintain a flux density of the magnetic field of at least about 4 millitesla (“mT”) to at least about 10 mT of the magnetic field contacting the droplet. In some embodiments, the controller is configured to maintain the magnet a distance of about 0 millimeters to about 15 millimeters from the surface. In some embodiments, the droplet is less than 30 microliters. In some embodiments, the droplet is less than 20 microliters. In some embodiments, the droplet is less than 10 microliters.

In some embodiments, at least 20% of the plurality of nucleic acid molecules comprise at least 100 kb. In some embodiments, at least 30% of the plurality of nucleic acid molecules comprise at least 100 kb. In some embodiments, at least 40% of the plurality of nucleic acid molecules comprise at least 100 kb. In some embodiments, at least 50% of the plurality of nucleic acid molecules comprise at least 100 kb. In some embodiments, at least 60% of the plurality of nucleic acid molecules comprise at least 100 kb. In some embodiments, at least 70% of the plurality of nucleic acid molecules comprise at least 100 kb. In some embodiments, at least 80% of the plurality of nucleic acid molecules comprise at least 100 kb. In some embodiments, at least 90% of the plurality of nucleic acid molecules comprise at least 100 kb.

In some embodiments, the system maintains at least 20% the plurality of nucleic acid molecules at 100 kb or more. In some embodiments, the system maintains the nucleic acid molecule at 100 kb or more. In some embodiments, the system maintains at least 20% the plurality of nucleic acid molecules at 100 kb or more. In some embodiments, the system maintains at least 30% the plurality of nucleic acid molecules at 100 kb or more. In some embodiments, the system maintains at least 40% the plurality of nucleic acid molecules at 100 kb or more. In some embodiments, the system maintains at least 50% the plurality of nucleic acid molecules at 100 kb or more. In some embodiments, the system maintains at least 60% the plurality of nucleic acid molecules at 100 kb or more. In some embodiments, the system maintains at least 70% the plurality of nucleic acid molecules at 100 kb or more. In some embodiments, the system maintains at least 80% the plurality of nucleic acid molecules at 100 kb or more. In some embodiments, the system maintains at least 90% the plurality of nucleic acid molecules at 100 kb or more.

In some embodiments, the distance is sufficient for immobilizing the one or more artifacts that are responsive to the magnetic field during one or more droplet operations. In some embodiments, the one or more droplet operations comprise agitation of the surface. In some embodiments, the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the one or more droplet operations comprise the droplet being stationary. In some embodiments, the one or more droplet operations comprise the droplet moving back and forth on the beads using electrowetting. In some embodiments, the one or more droplet operations comprise the droplet being heated or cooled. In some embodiments, the one or more droplet operations comprise a small aliquot from the droplet being removed. In some embodiments, the one or more droplet operations comprise droplet splitting. In some embodiments, the one or more droplet operations comprise the droplet being dispensed.

Another aspect of the instant disclosure is a device for processing a droplet, the device comprising: a surface configured to support a droplet; a magnet adjacent the surface, wherein the magnet is configured to provide a magnetic field contacting the droplet, wherein the droplet comprises one or more artifacts that are responsive to the magnetic field, and wherein the one or more artifacts that are responsive to the magnetic field comprise a nucleic acid molecule; a controller mechanically coupled to the magnet, wherein the controller is configured to displace the magnet proximate to the droplet and maintaining the nucleic acid molecule at 100 kb or more. In some embodiments, the magnet is displaced along an axis orthogonal to the surface. In some embodiments, the magnetic field contacting the droplet comprises a flux density of at least about 4 millitesla (“mT”) to at least about 10 mT. In some embodiments, the surface comprises an electrowetting array. In some embodiments, the one or more artifacts that are responsive to the magnetic field comprise a plurality of nucleic acid molecules. In some embodiments, the magnet is displaced along an axis parallel to the surface. In some embodiments, the controller is configured maintain a flux density of the magnetic field of at least about 4 millitesla (“mT”) to at least about 10 mT of the magnetic field contacting the droplet. In some embodiments, the controller is configured to maintain the magnet a distance of about 0 millimeters to about 15 millimeters from the surface. In some embodiments, the droplet is less than 30 microliters. In some embodiments, the droplet is less than 20 microliters. In some embodiments, the droplet is less than 10 microliters.

In some embodiments, at least 20% of the plurality of nucleic acid molecules comprise at least 100 kb. In some embodiments, at least 30% of the plurality of nucleic acid molecules comprise at least 100 kb. In some embodiments, at least 40% of the plurality of nucleic acid molecules comprise at least 100 kb. In some embodiments, at least 50% of the plurality of nucleic acid molecules comprise at least 100 kb. In some embodiments, at least 60% of the plurality of nucleic acid molecules comprise at least 100 kb. In some embodiments, at least 70% of the plurality of nucleic acid molecules comprise at least 100 kb. In some embodiments, at least 80% of the plurality of nucleic acid molecules comprise at least 100 kb. In some embodiments, at least 90% of the plurality of nucleic acid molecules comprise at least 100 kb.

In some embodiments, the distance is sufficient for immobilizing the one or more artifacts that are responsive to the magnetic field during one or more droplet operations. In some embodiments, the one or more droplet operations comprise agitation of the surface. In some embodiments, the one or more droplet operations comprise applying a voltage to at least one electrode of the one or more electrodes to manipulate the one or more reagent droplets, the sample droplet, or both. In some embodiments, the one or more droplet operations comprise applying a vibration to the one or more reagent droplets, the sample droplet, or both. In some embodiments, the one or more droplet operations comprise applying a vibration to the electrowetting array. In some embodiments, the one or more droplet operations comprise the droplet being stationary. In some embodiments, the one or more droplet operations comprise the droplet moving back and forth on the beads using electrowetting. In some embodiments, the one or more droplet operations comprise the droplet being heated or cooled. In some embodiments, the one or more droplet operations comprise a small aliquot from the droplet being removed. In some embodiments, the one or more droplet operations comprise droplet splitting. In some embodiments, the one or more droplet operations comprise the droplet being dispensed.

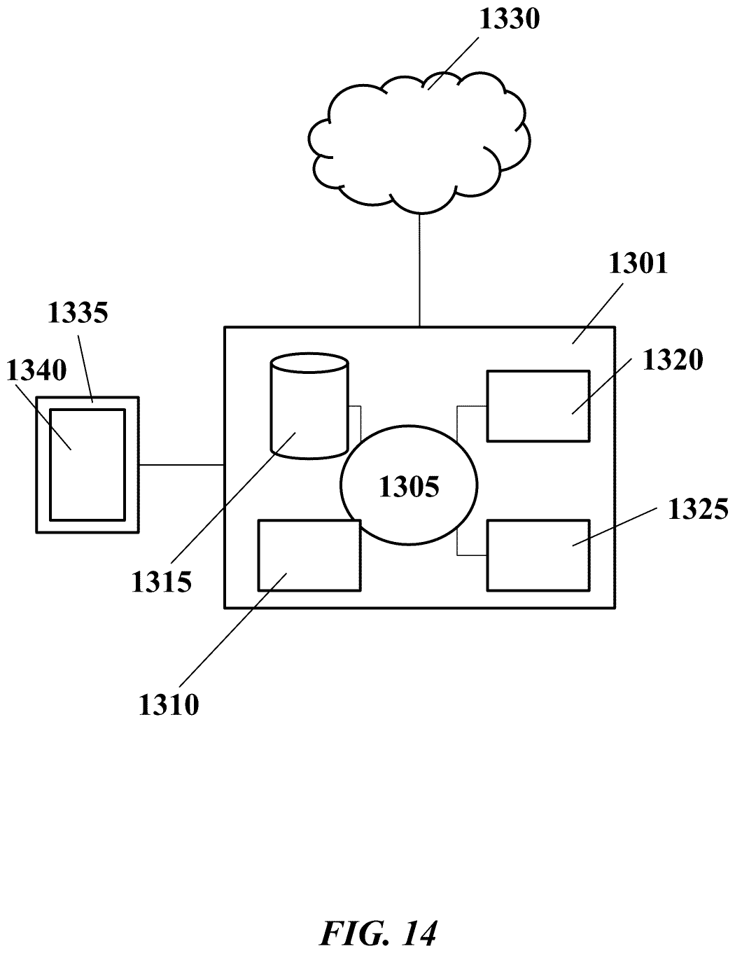

The present disclosure provides computer systems that are programmed to implement methods of the disclosure. FIG. 14 shows a computer system 1301 that is programmed or otherwise configured to manipulate a droplet, or a plurality thereof, on a system described herein. The computer system 1301 can regulate various aspects of sample manipulation of the present disclosure, such as, for example, droplet size, droplet volume, droplet position, droplet speed, droplet wetting, droplet temperature, droplet pH, beads in droplets, number of cells in droplets, droplet color, concentration of chemical material, concentration of biological substance, or any combination thereof. The computer system 1101 can be an electronic device of a user or a computer system that is remotely located with respect to the electronic device. The electronic device can be a mobile electronic device.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage, electronic display adapters, or any combination thereof. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet, extranet, or any combination thereof, or an intranet, extranet, or any combination thereof that is in communication with the Internet. The network 1330 in some cases is a telecommunication, data network, or any combination thereof. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1101 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet.

The computer system 1301 can communicate with one or more remote computer systems through the network 1330. For instance, the computer system 1301 can communicate with a remote computer system of a user (e.g., mobile electronic device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code, associated data, or any combination thereof that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code, data, or any combination thereof. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, information related to droplet manipulation, sample manipulation, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, provide additional liquid to a droplet, replace evaporated solvent of a droplet, map out a path for a droplet, or any combination thereof.

Video, input, and control of the system may be accessed through a web-based software application. User inputs through software may include, for example, droplet motion, droplet sizes, and images of the array, and user inputs may be recorded and stored in a cloud-based computing system. Stored user inputs may be accessed and retrieved in subsets or in entirety to inform machine-learning based algorithms. Droplet movement patterns may be recorded and analyzed for use in training navigation algorithms. Trained algorithms may be used for automation of droplet movement. Spatial fluid properties may be recorded and analyzed for use in training protocol optimization and generation algorithms. Trained algorithms may be used for optimizing biological and droplet movement protocols or in the generation of new biological and droplet movement protocols. Biological quality control techniques (e.g., amplification-based quantification methods, fluorescence-based, absorbance-based quantification, surface plasmon resonance methods, and capillary-electrophoretic methods to analyze nucleic acid fragment size) may be used to analyze the effectiveness of the workflows performed on the array. The data from these techniques may then be used as an input into machine learning algorithms to improve output. The process may be automated so that the system can iteratively improve the output.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1: Moving Magnet for Magnetic Bead-Assisted Separation

An electrowetting on dielectric (“EWOD”) device is provided as described in PCT/US2019/019954, PCT/US2020/048241, or PCT/US2022/046102, each of which is incorporated by reference in its entirety. A magnet is coupled to the EWOD device by a member configured to displace the magnet along an axis orthogonal to the surface of the EWOD device.

The EWOD device is used for magnetic bead assisted separation of DNA from cell lysate. The desired flux of the magnetic field exerted onto the magnetically responsive beads suspended within a droplet disposed onto the EWOD device is proportionate to the amount of sheering of DNA fragments that is tolerable for a given fragment size.

If very little or no shearing can be tolerated, the magnet is disposed closer to the surface of the EWOD device (e.g. 0 mm-10 mm). Manipulating the magnetic field in this manner results in increased clearance proximate to the surface of the EWOD device, enabling the presence of further actuators of droplet operations (e.g. vibrators, heaters, coolers, electrodes, etc.).

In addition to moving orthogonal to the surface of the EWOD device, the member is configured to dispose the magnet parallel to the surface of the EWOD device. The member is configured to dispose the magnet parallel to the surface of the EWOD device along both the x and y axes parallel to the surface of the EWOD device. This capability of the instantly described devices of having a magnet that can be disposed both orthogonal and parallel to the surface of the EWOD device enables users of the EWOD device to dispose the magnet in tandem with a sequence of electrode activations—thereby disposing the droplet itself on the surface of the EWOD device along the sequence—while also varying the flux density of the magnetic field exerted onto the magnetically responsive beads encapsulated in the droplet.

Precise control over the flux density of the magnetic field exerted onto the magnetically responsive beads encapsulated in the droplet during droplet operations enables a wide variety of assays and further droplet operations that can be utilized on the EWOD device.

What is claimed is:

1. A method of processing a droplet comprising:
a. Providing an electrowetting array, wherein said electrowetting array is adjacent to a magnet configured to apply a magnetic field to said electrowetting array in one or more of three directional axes;
b. Providing on said array a droplet comprising one or more artifacts that are responsive to said magnetic field;
c. Actuating said magnet in said one or more of three directional axes with respect to said array to separate said one or more artifacts from said droplet.

2. The method of claim 1, wherein said magnet is moved parallel to said array in the x direction.

3. The method of claim 1, wherein said magnet is moved parallel to said array in the y direction.

4. The method of claim 1, wherein said magnet is moved orthogonally to said array.

5. The method of claim 1, wherein the strength of said magnetic field is modulated.

6. The method of claim 5, wherein the strength of said magnetic field is increased.

7. The method of claim 5, wherein the strength of said magnetic field is decreased.

8. The method of claim 1, wherein said magnet is a permanent magnet.

9. The method of claim 1, wherein said magnet is an electromagnet or an electro-permanent magnet.

10. The method of claim 9, wherein said electromagnet or said electro-permanent magnet modulates the strength of said magnetic field in a time-dependent manner.

11. The method of claim 1, wherein said magnet is positioned above said electrowetting array.

12. The method of claim 11, wherein said magnet moves orthogonally with respect to said electrowetting array to separate said one or more artifacts from said droplet.

13. The method of claim 1, wherein said magnet is positioned below said electrowetting array.

14. A method of removing one or more artifacts from a droplet comprising providing a magnet configured to apply a magnetic field to said droplet and actuating said magnet with respect to said droplet to separate said one or more artifacts from said droplet, wherein said droplet is less than 40 microliters.

15. The method of claim 14, wherein said magnet is moved parallel to said array in the x direction.

16. The method of claim 14, wherein said magnet is moved parallel to said array in the y direction.

17. The method of claim 14, wherein said magnet is moved orthogonally to said array.

18. The method of claim 14, wherein the strength of said magnetic field is modulated.

19. The method of claim 18, wherein the strength of said magnetic field is increased.

20. The method of claim 18, wherein the strength of said magnetic field is decreased.

21. The method of claim 14, wherein said magnet is a permanent magnet.

22. The method of claim 14, wherein said magnet is an electromagnet or an electro-permanent magnet.

23. The method of claim 23, wherein said electromagnet or said electro-permanent magnet modulates the strength of said magnetic field in a time-dependent manner.

24. The method of claim 14, wherein said magnet is positioned above said electrowetting array.

25. The method of claim 23, wherein said magnet moves orthogonally with respect to said electrowetting array to separate said one or more artifacts from said droplet.

26. The method of claim 25, wherein said magnet is positioned below said electrowetting array.

27. The method of claim 14, wherein said droplet is less than 30 microliters.

28. The method of claim 27, wherein said droplet is less than 20 microliters.

29. The method of claim 28, wherein said droplet is less than 10 microliters.

30. A method for processing a droplet, the method comprising:
a. providing said droplet on a surface, wherein said surface is adjacent to a magnet configured to provide a magnetic field contacting said droplet, and wherein said droplet comprises one or more artifacts that are responsive to said magnetic field;
b. displacing said magnet proximal to said droplet, wherein said magnetic field contacting said droplet comprises a flux density of at least about 4 millitesla (“mT”) to at least about 10 mT,
thereby manipulating said one or more artifacts that are responsive to said magnetic field.

31. The method of claim 30, wherein said magnet is displaced along an axis orthogonal to said surface.

32. The method of any one of the preceding claims, wherein said magnet is displaced at a distance of about 0 millimeters to about 15 millimeters from said surface.

33. The method of any one of the preceding claims, wherein said surface comprises an electrowetting array.

34. The method of any one of the preceding claims, wherein said one or more artifacts that are responsive to said magnetic field comprise a nucleic acid molecule.

35. The method of any one of the preceding claims, wherein said one or more artifacts that are responsive to said magnetic field comprise a plurality of nucleic acid molecules.

36. The method of any one of the preceding claims, wherein said magnet is displaced along an axis parallel to said surface.

37. The method of any one of the preceding claims, wherein the method further comprises motioning said magnet and maintaining said flux density of at least about 4 millitesla (“mT”) to at least about 10 mT of said magnetic field contacting said droplet.

38. The method of any one of the preceding claims, wherein the method further comprises motioning said magnet and maintaining said distance of about 0 millimeters to about 15 millimeters from said surface.

39. The method of any one of the preceding claims, wherein said droplet is less than 30 microliters.

40. The method of any one of the preceding claims, wherein said droplet is less than 20 microliters.

41. The method of any one of the preceding claims, wherein said droplet is less than 10 microliters.

42. The method of any one of the preceding claims, wherein said nucleic acid molecule comprises at least 100 kb.

43. The method of any one of the preceding claims, wherein at least 20% of said plurality of nucleic acid molecules comprise at least 100 kb.

44. The method of any one of the preceding claims, wherein the method further comprises motioning said magnet and maintaining said nucleic acid molecule at 100 kb or more.

45. The method of any one of the preceding claims, wherein the method further comprises motioning said magnet and maintaining at least 20% said plurality of nucleic acid molecules at 100 kb or more.

46. The method of any one of the preceding claims, wherein said flux density is sufficient for immobilizing said one or more artifacts that are responsive to said magnetic field during one or more droplet operations.

47. The method of any one of the preceding claims, wherein said one or more droplet operations comprise agitation of said surface.

48. A method for processing a droplet, the method comprising:
a. providing said droplet on a surface, wherein said surface is adjacent to a magnet configured to provide a magnetic field contacting said droplet, and wherein said droplet comprises one or more artifacts that are responsive to said magnetic field;
b. displacing said magnet proximal to said droplet at a distance of about 0 millimeters to about 15 millimeters from said surface,
thereby manipulating said one or more artifacts that are responsive to said magnetic field.

49. The method of claim 30, wherein said magnet is displaced along an axis orthogonal to said surface.

50. The method of any one of the preceding claims, wherein said magnetic field contacting said droplet comprises a flux density of at least about 4 millitesla (“mT”) to at least about 10 mT.

51. The method of any one of the preceding claims, wherein said surface comprises an electrowetting array.

52. The method of any one of the preceding claims, wherein said one or more artifacts that are responsive to said magnetic field comprise a nucleic acid molecule.

53. The method of any one of the preceding claims, wherein said one or more artifacts that are responsive to said magnetic field comprise a plurality of nucleic acid molecules.

54. The method of any one of the preceding claims, wherein said magnet is displaced along an axis parallel to said surface.

55. The method of any one of the preceding claims, wherein the method further comprises motion said magnet and maintaining said flux density of at least about 4 millitesla (“mT”) to at least about 10 mT of said magnetic field contacting said droplet.

56. The method of any one of the preceding claims, wherein the method further comprises motioning said magnet and maintaining said distance of about 0 millimeters to about 15 millimeters from said surface.

57. The method of any one of the preceding claims, wherein said droplet is less than 30 microliters.

58. The method of any one of the preceding claims, wherein said droplet is less than 20 microliters.

59. The method of any one of the preceding claims, wherein said droplet is less than 10 microliters.

60. The method of any one of the preceding claims, wherein said nucleic acid molecule comprises at least 100 kb.

61. The method of any one of the preceding claims, wherein at least 20% of said plurality of nucleic acid molecules comprise at least 100 kb.

62. The method of any one of the preceding claims, wherein the method further comprises motioning said magnet and maintaining said nucleic acid molecule at 100 kb or more.

63. The method of any one of the preceding claims, wherein the method further comprises motioning said magnet and maintaining at least 20% said plurality of nucleic acid molecules at 100 kb or more.

64. The method of any one of the preceding claims, wherein the method further comprises motioning said magnet and maintaining said nucleic acid molecule at 100 kb or more.

65. The method of any one of the preceding claims, wherein the method further comprises motioning said magnet and maintaining at least 20% said plurality of nucleic acid molecules at 100 kb or more.

66. The method of any one of the preceding claims, wherein said distance is sufficient for immobilizing said one or more artifacts that are responsive to said magnetic field during one or more droplet operations.

67. The method of any one of the preceding claims, wherein said one or more droplet operations comprise agitation of said surface.

68. A method for processing a droplet, the method comprising:
a. providing said droplet on a surface, wherein said surface is adjacent to a magnet configured to provide a magnetic field contacting said droplet, wherein said droplet comprises one or more artifacts that are responsive to said magnetic field, and wherein said one or more artifacts that are responsive to said magnetic field comprise a nucleic acid molecule;
b. motioning said magnet and maintaining said nucleic acid molecule at 100 kb or more,
thereby manipulating said one or more artifacts that are responsive to said magnetic field.

69. The method of claim 30, wherein said magnet is displaced along an axis orthogonal to said surface.

70. The method of any one of the preceding claims, wherein said magnetic field contacting said droplet comprises a flux density of at least about 4 millitesla (“mT”) to at least about 10 mT.

71. The method of any one of the preceding claims, wherein said surface comprises an electrowetting array.

72. The method of any one of the preceding claims, wherein said one or more artifacts that are responsive to said magnetic field comprise a plurality of nucleic acid molecules.

73. The method of any one of the preceding claims, wherein said magnet is displaced along an axis parallel to said surface.

74. The method of any one of the preceding claims, wherein the method further comprises motion said magnet and maintaining said flux density of at least about 4 millitesla (“mT”) to at least about 10 mT of said magnetic field contacting said droplet.

75. The method of any one of the preceding claims, wherein the method further comprises motioning said magnet and maintaining said distance of about 0 millimeters to about 15 millimeters from said surface.

76. The method of any one of the preceding claims, wherein said droplet is less than 30 microliters.

77. The method of any one of the preceding claims, wherein said droplet is less than 20 microliters.

78. The method of any one of the preceding claims, wherein said droplet is less than 10 microliters.

79. The method of any one of the preceding claims, wherein at least 20% of said plurality of nucleic acid molecules comprise at least 100 kb.

80. The method of any one of the preceding claims, wherein the method further comprises maintaining at least 20% said plurality of nucleic acid molecules at 100 kb or more.

81. The method of any one of the preceding claims, wherein the method further comprises motioning said magnet and maintaining said nucleic acid molecule at 100 kb or more.

82. The method of any one of the preceding claims, wherein the method further comprises motioning said magnet and maintaining at least 20% said plurality of nucleic acid molecules at 100 kb or more.

83. The method of any one of the preceding claims, wherein said distance is sufficient for immobilizing said one or more artifacts that are responsive to said magnetic field during one or more droplet operations.

84. The method of any one of the preceding claims, wherein said one or more droplet operations comprise agitation of said surface.

85. A system for processing a droplet, the system comprising:
a. a surface configured to support a droplet;
b. a magnet adjacent said surface, wherein said magnet is configured to provide a magnetic field contacting said droplet, wherein said droplet comprises one or more artifacts that are responsive to said magnetic field, and wherein said one or more artifacts that are responsive to said magnetic field comprise a nucleic acid molecule;
c. a controller mechanically coupled to said magnet, wherein said controller is configured to displace said magnet proximate to said droplet and maintaining said nucleic acid molecule at 100 kb or more.

86. The system of claim 30, wherein said magnet is displaced along an axis orthogonal to said surface.

87. The system of any one of the preceding claims, wherein said magnetic field contacting said droplet comprises a flux density of at least about 4 millitesla (“mT”) to at least about 10 mT.

88. The system of any one of the preceding claims, wherein said surface comprises an electrowetting array.

89. The system of any one of the preceding claims, wherein said one or more artifacts that are responsive to said magnetic field comprise a plurality of nucleic acid molecules.

90. The system of any one of the preceding claims, wherein said magnet is displaced along an axis parallel to said surface.

91. The system of any one of the preceding claims, wherein said controller is configured maintain a flux density of said magnetic field of at least about 4 millitesla (“mT”) to at least about 10 mT of said magnetic field contacting said droplet.

92. The system of any one of the preceding claims, wherein said controller is configured to maintain said magnet a distance of about 0 millimeters to about 15 millimeters from said surface.

93. The system of any one of the preceding claims, wherein said droplet is less than 30 microliters.

94. The system of any one of the preceding claims, wherein said droplet is less than 20 microliters.

95. The system of any one of the preceding claims, wherein said droplet is less than 10 microliters.

96. The system of any one of the preceding claims, wherein at least 20% of said plurality of nucleic acid molecules comprise at least 100 kb.

97. The system of any one of the preceding claims, wherein the system maintains at least 20% said plurality of nucleic acid molecules at 100 kb or more.

98. The system of any one of the preceding claims, wherein the system maintains said nucleic acid molecule at 100 kb or more.

99. The system of any one of the preceding claims, wherein the system maintains at least 20% said plurality of nucleic acid molecules at 100 kb or more.

100. The system of any one of the preceding claims, wherein said distance is sufficient for immobilizing said one or more artifacts that are responsive to said magnetic field during one or more droplet operations.

101. The system of any one of the preceding claims, wherein said one or more droplet operations comprise agitation of said surface.

102. A device for processing a droplet, the device comprising:
a. a surface configured to support a droplet;
b. a magnet adjacent said surface, wherein said magnet is configured to provide a magnetic field contacting said droplet, wherein said droplet comprises one or more artifacts that are responsive to said magnetic field, and wherein said one or more artifacts that are responsive to said magnetic field comprise a nucleic acid molecule;
c. a controller mechanically coupled to said magnet, wherein said controller is configured to displace said magnet proximate to said droplet and maintaining said nucleic acid molecule at 100 kb or more.

103. The device of claim 30, wherein said magnet is displaced along an axis orthogonal to said surface.

104. The device of any one of the preceding claims, wherein said magnetic field contacting said droplet comprises a flux density of at least about 4 millitesla (“mT”) to at least about 10 mT.

105. The device of any one of the preceding claims, wherein said surface comprises an electrowetting array.

106. The device of any one of the preceding claims, wherein said one or more artifacts that are responsive to said magnetic field comprise a plurality of nucleic acid molecules.

107. The device of any one of the preceding claims, wherein said magnet is displaced along an axis parallel to said surface.

108. The device of any one of the preceding claims, wherein said controller is configured maintain a flux density of said magnetic field of at least about 4 millitesla (“mT”) to at least about 10 mT of said magnetic field contacting said droplet.

109. The device of any one of the preceding claims, wherein said controller is configured to maintain said magnet a distance of about 0 millimeters to about 15 millimeters from said surface.

110. The device of any one of the preceding claims, wherein said droplet is less than 30 microliters.

111. The device of any one of the preceding claims, wherein said droplet is less than 20 microliters.

112. The device of any one of the preceding claims, wherein said droplet is less than 10 microliters.

113. The device of any one of the preceding claims, wherein at least 20% of said plurality of nucleic acid molecules comprise at least 100 kb.

114. The device of any one of the preceding claims, wherein said distance is sufficient for immobilizing said one or more artifacts that are responsive to said magnetic field during one or more droplet operations.

115. The device of any one of the preceding claims, wherein said one or more droplet operations comprise agitation of said surface.
